(19) United States
(12) Patent Application Publication
(10) Pub. No.: US 2010/0278923 A1
(43) Pub. Date: Nov. 4, 2010

(54) Functional nanoparticle-based antibiotics and preparation method thereof

(75) Inventors: Yu-Chie Chen, Hsinchu (TW); Wei-Chieh Huang, Hualien (TW)

Correspondence Address: BACON & THOMAS, PLLC, 625 SLATERS LANE, FOURTH FLOOR, ALEXANDRIA, VA, 22314-1176, US

(73) Assignee: National Chiao Tung University, Hsinchu (TW)

(21) Appl. No.: 12/232,279

(22) Filed: Sep. 15, 2008

(30) Foreign Application Priority Data
Feb. 27, 2008 (TW) 097106729

(51) Int. Cl. A61K 9/14; A61K 38/14; A61P 31/04

(52) U.S. Cl. 424/490; 514/3.1; 977/773; 977/906; 977/810; 977/831

(57) ABSTRACT

The present invention is related to a novel functional nanoparticle-based antibiotics and preparation method thereof, especially related to an antibiotics-modified nanoparticle. The functional nanoparticle-based antibiotics according to the present invention can be used as the affinity probes and the photothermal agents to effectively inhibit the cell growth of pathogenic bacteria under NIR irradiation.

### BACKGROUND OF THE INVENTION

1. Field of the Invention

The present invention relates to a novel functional nanoparticle-based antibiotics and preparation method thereof, especially relates to an antibiotics-modified nanoparticle.

2. Description of the Prior Art

No matter the internal medicine therapy or surgery, the antibiotics are usually used to prevent the infection of pathogenic bacteria. The so-called antibiotics are the materials extracted from the fungi originally, which can kill bacteria or inhibit the cell growth of bacteria. The penicillin is the first antibiotic which is applied extensively in human medical history. Its discovery is a great breakthrough to the therapy of disease. Many incurable diseases would be able to be cured by this antibiotic, so it is nearly considered as a catholicon.

At present, there are nearly more than 1,000 kinds of antibiotics that can kill the bacteria effectively, including the antibiotics extracted from the cultivation liquid of microorganism and various synthetic or semi-synthetic products. According to the statistics of Department of Health of Taiwan, R.O.C., it is known that about 1,000 kinds of antibiotics that are used frequently in clinic. But due to the abuse of the antibiotics, most bacteria evolve into the strains with resistance to the action of medicine gradually, so it is necessary to develop more effective medicine to inhibit the cell growth of many kinds of bacteria and prevent the threat from the infection of pathogenic bacteria.

At present, when the bacteria with drug-resistant are encountered clinically, the so-called last line medicine—vancomycin—will be used, because its medical effect is relatively strong, so it will only be used when other antibiotics are invalid. Generally speaking, the vancomycin is mainly used to inhibit the cell growth of Gram-positive bacteria, such as Enterococcus spp., Streptococci spp., and Staphylococci spp., etc. It can destroy the synthesis of cell wall, make new cell wall loose and unable to resist the strong osmotic pressure inside bacterium body, cause the rupture of bacterium body finally and achieve the sterilization purpose. But under the abuse condition of antibiotics clinically, the last defense line of vancomycin has always been used earlier, so many bacteria such as vancomycin-resistant Enterococcus (VRE) can resist the vancomycin. It will make much more serious problem of clinical infection, and cause the secret worry on the prevention of infectious diseases.

According to the report of World Health Organization on the problem about the drug-resistance of pathogenic bacteria, if the abuse problem of antibiotics is not faced seriously, the curable diseases such as throat disease, ear inflammation, malaria, and pulmonary tuberculosis, etc. will not be able to be cured by any medicine in the future. Only ten to twenty years left for people to use the existing antibiotics to treat the infectious diseases.

Therefore, upon seeing the serious problem for the abuse of antibiotics and the drug-resistance of bacteria, the present invention provides a novel functional nanoparticle-based antibiotics and preparation method thereof. The brief illustration of the invention is described as follows.

### SUMMARY OF THE INVENTION

The present invention provides a novel functional nanoparticle-based antibiotics and preparation method thereof, especially an antibiotics-modified nanoparticle, in order to solve the drug-resistance of bacteria clinically. According to the preparation method of the present invention, the nanoparticle, which surface is modified and fixed by the antibiotics molecules, is capable of absorbing near infrared radiation (NIR), recognizing the pathogenic bacteria, and inhibiting the cell growth of bacteria under NIR irradiation as affinity probes and photothermal agents. Therefore, the antibiotics of the present invention can inhibit cell growth of multiple drug-resistance pathogens.

In one aspect, the present invention provides a novel functional nanoparticle-based antibiotics, including a nanoparticle and an antibiotics, which can recognize bacteria, and the antibiotics is fixed on a surface of the nanoparticle.

According to an embodiment of the present invention, the nanoparticle can absorb NIR.

Preferably, the nanoparticle is a metal nanoparticle, a nanoparticle with a core-shell structure, or an electroceramic nanocomposite.

More preferably, the nanoparticle is a gold nanoparticle or a silica nanoparticle coated with a gold shell.

According to another embodiment of the present invention, the antibiotics can recognize Gram-positive bacteria or Gram-negative bacteria.

Preferably, the antibiotics can recognize vancomycin-resistant bacteria.

In another aspect, the present invention provides a preparation method for a functional nanoparticle-based antibiotics, including providing a nanoparticle solution, preparing an antibiotics solution, and reacting the nanoparticle solution with the antibiotics solution, therefore the antibiotics is fixed on a surface of the nanoparticle by a covalent bond.

According to an embodiment of the present invention, the nanoparticle is a metal nanoparticle, a nanoparticle with a core-shell structure, or an electroceramic nanocomposite.

Preferably, the nanoparticle is a gold nanoparticle or a silica nanoparticle coated with a gold shell.

According to another embodiment of the present invention, the antibiotics is vancomycin.

Preferably, the vancomycin is the vancomycin dimer bearing a disulfide linkage.

In another aspect, the present invention provides a method of using the antibiotics of the present invention to treat bacteria infection, including administrating the antibiotics with an effective dose to a subject in need thereof, and exposing the subject under a near infrared radiation light source.

According to an embodiment of the present invention, the near infrared radiation light source is a laser.

In another aspect, the present invention provides a pharmaceutical composition for treating bacteria infection, including the antibiotics of the present invention and a pharmaceutically acceptable carrier thereof.

Preferably, the carrier is a solution, an excipient or an adhesive agent.

In another aspect, the present invention provides a method of using the antibiotics of the present invention to treat bacteria infection, comprising administrating the antibiotics with an effective dose to a subject in need thereof, and exposing the subject under a near infrared radiation light source.

In order to solve drug-resistant problems of bacteria to antibiotics, the present invention combines the antibiotics and photothermal therapy (PTT) or photodynamic therapy (PDT), to recognize the bacteria through the antibiotics, and use photothermal therapy to kill bacteria, and increase the effect of bacteria inhibition.

The so-called NIR photothermal therapy is to use the nanoparticles or organic molecules to get significant heat effect after absorbing the energy in a specific range of wavelength, so it can raise the temperature of cell at local tissue, therefore destroy the cell or inhibit the cell growth. As for the nanomaterials studied at present, the one with small cytotoxicity and utilized most generally is gold nanoparticles. But there are some shortcomings which have to be improved. For example, most photothermal therapy with the utilization of laser as light source focus on inhibiting the growth of cancer cells, but there are relevant researches or applications which inhibit or kill the pathogenic bacteria with higher heat resistance. In order to solve the drug-resistant problem of bacteria, the present invention combines the nanoparticles with NIR absorption ability and the antibiotics with bacteria recognition ability to prepare a novel functional nanoparticle-based antibiotics.

In recent years, the development of photothermal therapy is different from the small organic molecule with light absorption property. A gold nanoparticle is used gradually because it has excellent absorption property around 520 nm of visible light. Or a silica nanoparticle coated with a gold shell with NIR absorption property is synthesized. The nanoparticles absorb laser energy from the visible the visible light with fixed wavelength and output power or NIR Agar laser (Nd:YAG laser) in short time (ns). The heat is transferred to the surface to damage or kill the target cell identified by the nanoparticle due to high temperature and pressure change. The research in this respect is usually to modify the nanoparticle surface by the specific antibody with recognizing nature of surface antigen of cancer cell first, or the specific protein and immunoglobulin with recognizing nature of bacteria. Therefore these nanoparticles used as photothermal agent can approach the surface of target cell through the specific recognizing process, so as to raise the efficiency of photothermal therapy. But its shortcoming lies in that the wavelength of visible light is easy to be absorbed by the tissue itself, so if the photothermal therapy under several centimeters of the epidermis wanted to be achieved, NIR with better permeating rate should be selected. At the same time, when the antibiotics with bacteria recognition property is applied together, it will be able to raise the applicability of functional nanoparticle-based antibiotics of the present invention.

The nanoparticle used in the present invention includes but not limits to metal nanoparticles, nanoparticles with core-shell structures, or electroceramic nanocomposites. Among them, the most applicable one are gold nanoparticles. Gold nanoparticles are widely applied in biology and analysis. They have some advantages such light stability, lower biological toxicity, and easy to be modified by antibodies and proteins, etc. Furthermore, by changing the size, shape or ratio of core/shell thickness, the surface plasma resonance of nanoparticles will generate the phenomenon of red displacement, and it even can reach the absorption range of NIR (700 nm˜1300 nm). The most common method is to use the surfactants or polymers as coating agents to synthesize the nanoparticle with NIR absorption ability. But these chemical materials have cell toxicity to the organism, and the surface of nanoparticle is difficult to be modified by biological molecules. Another kind of method is photochemical synthesis, but it still needs to use surfactants, and the irradiation time of light is as long as 12˜48 hours, so it is really inconvenient technically.

In addition, except gold nanoparticles, nanoparticles with core-shell structures, or electroceramic nanocomposites can also be used for the functional nanoparticle-based antibiotics of the present invention. As for a silica nanoparticle coated with a gold shell, a nanoparticle with a core-shell structure can be manufactured by modifying the positive charge on the surface of the silica nanoparticle, then the gold seeds with negative charge are attracted to the surface. Finally, the reduction reaction is conducted, and the gold shell coats on the surface of silica nanoparticle. When the same method is processed, an electroceramic nanoparticle is synthesized by absorbing magnetic iron oxide particles before absorbing the seed. In this kind of core-shell nanocomposite, when the thickness of the core (silica) and shell (gold) reach a specific ratio, it can absorb the wavelength of NIR. In a preferred embodiment, the thickness of gold shell is also in nano scale.

In summary, the best photothermal agent should be a nanoparticle with NIR absorption ability, and its surface is easy to be modified by biological recognizing molecules. The functional nanoparticle-based antibiotics prepared according to the method of the present invention, which has both above-mentioned advantages, can prevent the drug-resistant problem of bacteria through the enforcement of photothermal therapy. As for the patient infected at the initial stage with the infection and spreading of the pathogenic bacteria, if the photothermal therapy can be effectively used to inhibit the cell growth of the bacteria fast, it will have the opportunity to avoid the complication of bacteraemia or septicaemia.

The antibiotics-modified (e.g., vancomycin) nanoparticle provided by the present invention can recognize several pathogenic bacteria, including non-drug-resistant Staphylococcus aureus, Streptococcus pyogenes, Escherichia coli (urinary tract infections, UTI, E. coli O157:H7), Acinetobacter baumannii, vancomycin-resistant Enterococcus (VRE), methicillin-resistant S. aureus (MRSA), and pan-drug resistant A. baumannii (PDRAB). And the antibiotics-modified nanoparticle according to the present invention can kill most bacteria (>99%) under low-energy (200 mW/cm²) NIR laser irradiation in short time (such as several minutes). Due to the wavelength of NIR is not easy to be absorbed by tissue fluids, the photothermal therapy designed in the present invention can reach deeper tissue. Upon comparing the disinfecting effect with the present visible light laser, the nanoparticle modified by vancomycin of the present invention has better bacteria recognition ability, and the laser energy is lower with better penetration. If it is used in the clinic further, it can inhibit the cell growth of bacteria, which can provide faster and more effective auxiliary therapy way to the infected patient.

Moreover, the antibiotics of the prevent invention are further used as probes to recognize surfaces of pathogenic bacteria. Therefore, as used herein, “antibiotics” includes, but not limited to, natural antibiotics; modified natural antibiotics; synthesized antibiotics; immunoglobulins or fragments thereof (see, Ho et al., Anal. Chem. 2004, 76, 7162-7168; and Chen et al., small 2008, 4, (4), 485-491); polyclonal or monoclonal antibodies (Norman et al., Nano Lett. 2008, 8, 302-306); pigeon ovalbumin, which can recognize uropathogenic Escherichia coli (Liu et al., Anal. Chem. 2008, 80, 5425-5432); aptamers, which are artificial oligonucleotides and can bind to different biomolecules (So et al., small 2008, 4, (2), 197-201); inorganic metal oxides, for example, Titania (TiO₂), Zirconia (ZrO₂), Tantalum oxide (Ta₂O₅), Niobium oxide (Nb₂O₅), Zinc oxide (ZnO) or Alumina (Al₂O₃) (see, Chen and Chen, Anal. Chem. 2005, 77, 5912-5919; Lin et al., Anal. Chem. 2006, 78, 6873-6878; Chen et al., J. Proteome Res. 2007, 6, 316-325; Lo et al., J. Proteome Res. 2007, 6, 887-893); and the like. Each mentioned publication is incorporated herein by reference.

The category of antibiotics used for the functional nanoparticle-based antibiotics of the present invention is also not limited. Any antibiotics which can be modified on the surface of nanoparticles by either covalent bonds or non-covalent bonds will be suitable to be applied. For example, the vancomycin with sulfur group is used to form sulfur-gold covalent bond on the surface of gold nanoparticles, to prepare the functional nanoparticle-based antibiotics of the present invention.

The nanoparticle of the present invention has larger specific surface area. After the surface being modified by the antibiotics with bacteria recognition ability (such as vancomycin), the nanoparticle will be able to recognize the pathogenic bacteria effectively. When NIR laser is used as the light source, and the nanoparticle is used as the medium to adsorb the energy, it can kill most bacteria due to the relief of heat on the surface of bacteria in short time.

### BRIEF DESCRIPTION OF THE DRAWINGS

The foregoing aspects and many of the attendant advantages of this invention will become more readily appreciated as well becomes better understood by reference to the following detailed description, when taken in conjunction with the accompanying drawings, wherein:

FIG. 1 shows the flow chart for the preparation of the functional nanoparticle-based antibiotics of the present invention.

FIG. 2 shows the ultraviolet-visible light absorption spectrum of gold nanoparticles before and after the modification of antibiotics of the present invention.

FIG. 3 shows the ultraviolet absorption spectrum of antibiotics before and after the modification of gold nanoparticles of the present invention.

FIG. 4(A), FIG. 4(B), FIG. 4(C), and FIG. 4(D) show the electron microscopy pictures for the recognition of various bacteria strains by the functional nanoparticle-based antibiotics of the present invention.

FIG. 5 shows the pictures of the culture plates after irradiating by NIR in accordance with the embodiment of the present invention.

### DESCRIPTION OF THE PREFERRED EMBODIMENT

The following is a description of the present invention. The invention firstly will be described with reference to one exemplary structure. Some variations will then be described as well as advantages of the present invention. A preferred method of fabrication will then be discussed. An alternate, asymmetric embodiment will then be described along with the variations in the process flow to fabricate this embodiment.

The present invention provides a novel functional nanoparticle-based antibiotics and preparation method thereof. The present invention will be understood sufficiently by the description of the following embodiment, and those skilled in the art can finish it accordingly. But the scope of this invention shall not limited by the following embodiment.

### Embodiment 1

### The synthesis of the Gold Nanoparticle with NIR Adsorption

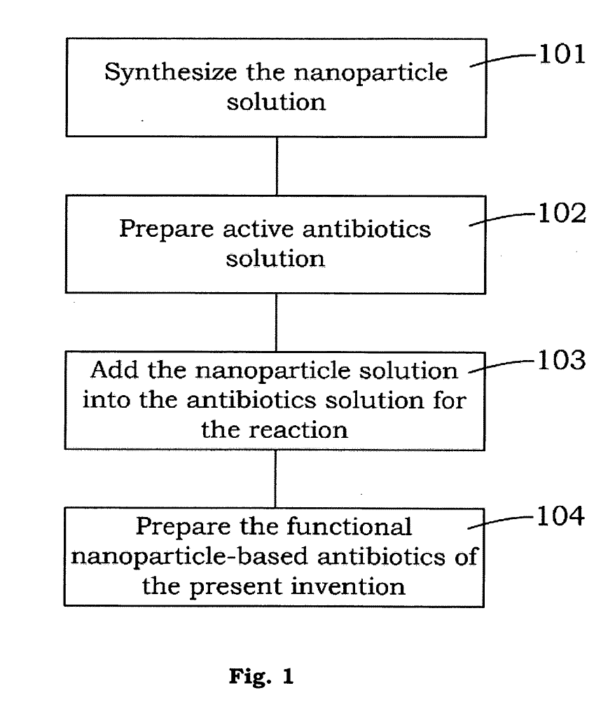

FIG. 1 shows the flow chart for the synthesis of functional nanoparticle of the present invention. Please refer to Step 101: First, 0.0402 g of sodium oxalate (Na₂C₂O₄) is dissolved into 3 ml of deionized water to form 0.1 M oxalic acid solution. 65 μl of the solution is taken and dropped into a quartz bottle, which is covered a layer of tinfoil on the outside of quartz bottle to avoid the irradiation of light, with 9.7 ml of deionized water. The solution is shaken slightly, and then added dropwise into gold chloride trihydrate (HAuCl₄) solution (0.01 M, 0.3 ml). The bottle is sealed with the parafilm, then put under the ultraviolet light (306 nm of wavelength, 8 W), and irradiated for 50 minutes at 250 rpm/min to complete the preparation of gold nanoparticles. Finally, the solution is isolated by the centrifuge for 20 minutes at 2,500 rpm to remove the supernatant; 0.2 ml gold nanoparticle solution is kept for the further application.

### Embodiment 2

### The Modification of Gold Nanoparticles by Vancomycin

As shown in Step 102 of FIG. 1, referring to the method of Sundram et al. to prepare the solution of vancomycin dimmers bearing a disulfide linkage (J. Am. Chem. Soc. 1996, 118, 13107-13108). 0.6 ml vancomycin dimer solution (0.1 mg/ml) is added into a 20 ml glass bottle. The gold nanoparticle solution (0.2 ml) is added dropwise into the bottle slowly. The bottle is then slightly shaken for 12 hours at 50 rpm/min (as shown in Step 103 of FIG. 1). After that, the vancomycin-modified gold nanoparticle solution (Au@van) is separated by the centrifuge for 20 minutes at 6,000 rpm to remove the supernatant, 0.6 ml of deionized water is used for rinsing, and then the solution is isolated by the centrifuge at 6,000 rpm again. Finally, the precipitate of Au@van is resuspended in 0.2 ml of deionized water to complete the preparation of vancomycin-modified gold nanoparticles (as shown in Step 104 of FIG. 1).

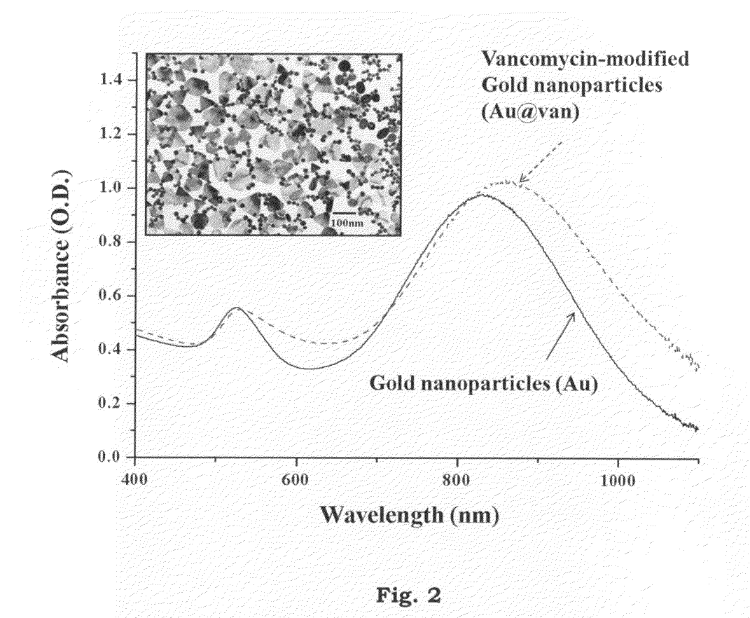

FIG. 2 shows the ultraviolet (UV) absorption spectrum of gold nanoparticles before and after modified by vancomycin. As shown in FIG. 2, after the gold nanoparticle modified by vancomycin with sulfur-gold covalent bond, the maximum absorption wavelength is increased. It means the maximum absorption wavelength is influenced by the resonance frequency of surface free electron after gold nanoparticle is modified by vancomycin.

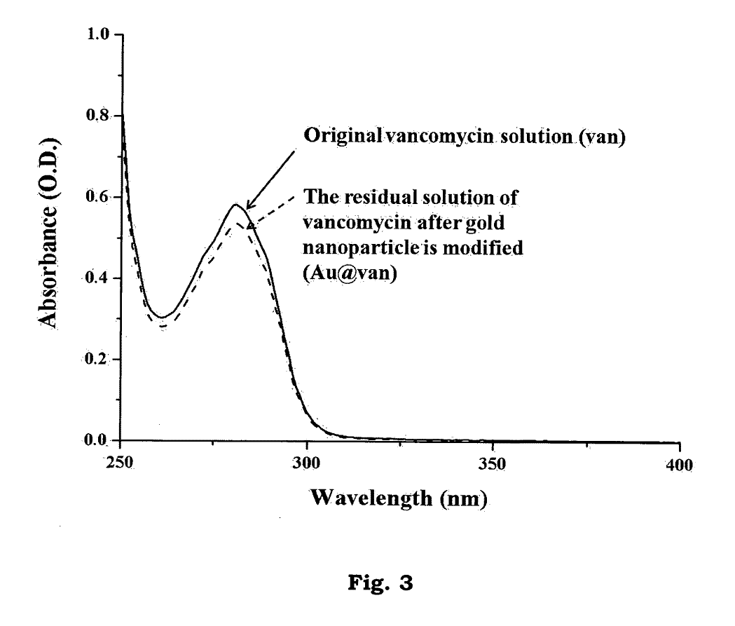

FIG. 3 shows the ultraviolet (UV) absorption spectrum of original vancomycin solution, and the residual solution of vancomycin after gold nanoparticle is modified by vancomycin. Generally, the maximum absorption wavelength of vancomycinis is 280 nm. Therefore the decrease amount of this value can be substituted into the calibration curve of original vancomycin, to calculate how many mg of gold nanoparticles are modified by the certain amount moles of vancomycin. In this embodiment, about 1 mg of gold nanoparticle is modified by 30 nmole of vancomycin.

Therefore, as shown in FIG. 2 and FIG. 3, the vancomycin can be used to modify gold nanoparticles to prepare a functional nanoparticle-based antibiotics of the present invention.

### Embodiment 3

### Test for the Survival Rate of Bacteria

First, use the inoculating ring to remove the bacteria colony from the cultivation plate into the Tryptic Soy Broth (TSB) medium (12 g of TSB powder and 2 g of yeast are dissolved in 400 ml of deionized water, sterilized at high temperature and high pressure, and then cooled down to the room temperature). The inoculated medium is cultivated at 37□ and 150 rpm for 8 hours. Some bacteria solution is taken out, diluted with the sterilized TSB to the light absorption value of O.D.₆₀₀=1. Then, 1 ml of solution is put into the Eppendorf tube. The solution is centrifuged for 5 minutes at 2,100 rpm. After the supernatant is removed, 1 ml of sterilized PBS buffer solution (about 0.1 mM) is added. The surplus TSB is washed out by vortex, and this washing step is repeated twice. Finally, the bacteria solution is resuspended in 1 ml of PBS buffer solution, where the PBS buffer solution is prepared by mixing 40.5 ml, 0.2 M of Sodium hydrogen phosphate (Na₂HPO₄) and 9.5 ml, 0.2 M of Sodium dihydrogen phosphate (NaH₂PO₄) (pH=7.4), and then diluting to 0.1 mM.

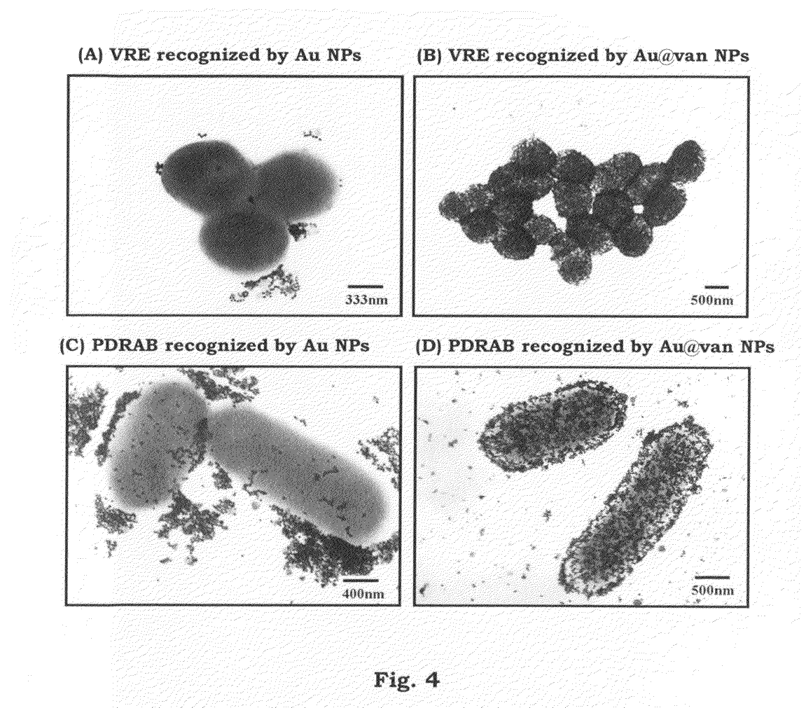

The 8 kinds of bacteria solutions, each O.D.₆₀₀=1, are serial diluted to a suitable ratio (10⁴˜10⁵ cells/mL) with PBS buffer solution, respectively. Each 55 μl of bacteria solution is then put into the Eppendorf tube, respectively, with 0.1 ml of Au@van solution at room temperature for 15 minutes, so that the vancomycin-modified gold nanoparticles can recognize the target bacteria. FIG. 4(A), FIG. 4(B), FIG. 4(C), FIG. 4(D) show the electron microscopy pictures for the recognition of vancomycin-resistant Enterococcus (VRE) and Pan-drug resistant A. baumannii (PDRAB) by the vancomycin-modified gold nanoparticles. It is clearly shown that the vancomycin-modified gold nanoparticles can recognize both bacteria on the surface effectively.

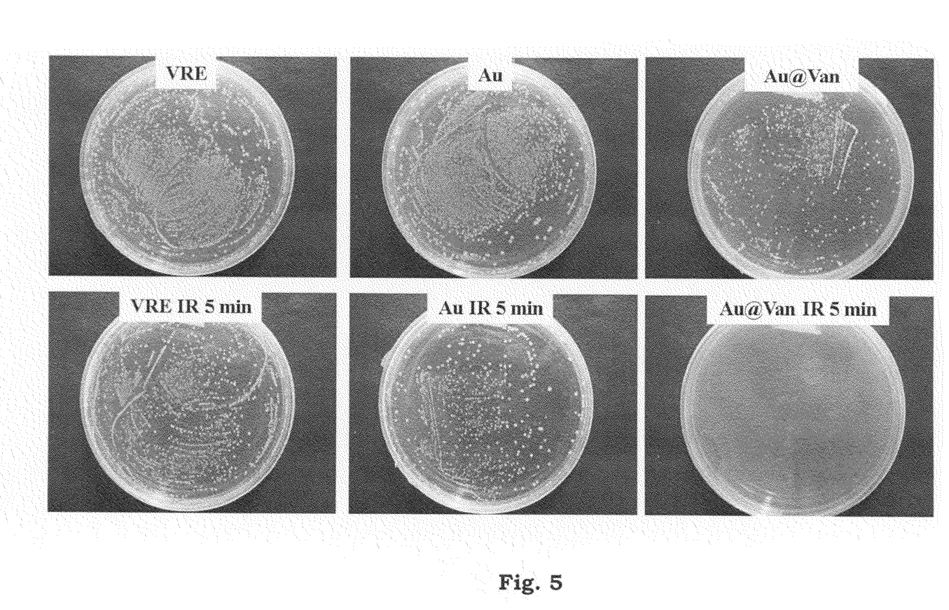

The Eppendorf tubes are moved to 4.5 cm away from the laser light source and irradiated for 5 minutes. Finally, the solution is taken out from the Eppendorf tube via pipettes, inoculated on the culture plate, and cultivated for 8-42 hours at 37□. The results are shown in FIG. 5. Compare to the control group, it is found that the vancomycin-modified gold nanoparticles can inhibit the cell growth of bacteria.

The bacteria colony number of residual bacteria is calculated. Upon comparing the results of control group (only cultivated in PBS buffer solution) and un-modified gold nanoparticle group, the survival rate of bacteria can be calculated, and the results are shown in Table 1.

TABLE 1
Bacteria inhibition results for the functional
nanoparticle-based of the present invention antibiotics

| | Survival rate (N/N₀ * %) | | |
|---|---|---|---|
| | | Gold | Vancomycin-modified |
| | Control | nanoparticle | gold nanoparticle |
| Bacteria strains | group | (Au) | (Au@van) |
| Streptococcus | 72.14 | 5.960 | 0.445 |
| pyogenes | | | |
| Staphylococcus | 88.57 | 1.102 | 0.049 |
| aureus | | | |
| Vancomycin-resistant | 82.25 | 1.436 | 0.236 |
| Enterococcus (VRE) | | | |
| Escherichia coli (UTI) | 97.72 | 0.803 | 0.102 |
| Escherichia coli | 80.00 | 0.210 | 0.016 |
| (O157:H7) | | | |
| Acinetobacter | 85.01 | 1.405 | 0.384 |
| baumannii | | | |
| Pan-drug resistant | 77.56 | 3.239 | 0.554 |
| A. baumannii | | | |
| (PDRAB) | | | |
| Methicillin-resistant | 91.95 | 0.625 | 0.056 |
| S. aureus (MRSA) | | | |

In Table 1, No represents the original bacteria number, and N represents the survived bacteria number. From the results of Table 1, it is found that although both gold nanoparticles and vancomycin-modified gold nanoparticles can inhibit the cell growth of bacteria, but the bacteria inhibition effect of the vancomycin-modified gold nanoparticles is highly significant.

The functional nanoparticle-based antibiotics provided by the present invention have the NIR absorption property, and the absorption efficiency is very good, so that the local temperature of the gold nanoparticle can be raised significantly. The surface of gold nanoparticle is modified by the nanoparticle with bacteria recognition ability. Due to the gold nanoparticle of the present invention has large specific surface area, so the functional nanoparticle-based antibiotics of the present invention can be developed to a pharmaceutical agent to recognize various pathogenic bacteria and drug-resistant bacteria under the action of vancomycin. In the same time, it can generate sufficient heat under the irradiation of NIR in very short time (less than 5 minutes), to reach more than 99% death rate of bacteria, so it has very good development potential in clinical therapy.

It is understood that various other modifications will be apparent to and can be readily made by those skilled in the art without departing from the scope and spirit of this invention. Accordingly, it is not intended that the scope of the claims appended hereto be limited to the description as set forth herein, but rather that the claims be construed as encompassing all the features of patentable novelty that reside in the present invention, including all features that would be treated as equivalents thereof by those skilled in the art to which this invention pertains.

What is claimed is:

1. A functional nanoparticle-based antibiotics, comprising:
a nanoparticle; and
an antibiotics to recognize bacteria;
wherein the antibiotics is fixed on a surface of the nanoparticle.

2. The antibiotics according to claim 1, wherein the nanoparticle is capable of absorbing near infrared radiation.

3. The antibiotics according to claim 2, wherein the nanoparticle is a metal nanoparticle, a nanoparticle with a core-shell structure, or an electroceramic nanocomposite.

4. The antibiotics according to claim 3, wherein the nanoparticle is a gold nanoparticle or a silica nanoparticle coated with a gold shell.

5. The antibiotics according to claim 1, wherein the antibiotics is capable of recognizing Gram-positive bacteria or Gram-negative bacteria.

6. The antibiotics according to claim 1, wherein the antibiotics is capable of recognizing vancomycin-resistant bacteria.

7. A method of preparing a functional nanoparticle-based antibiotics, comprising:
providing a nanoparticle solution;
preparing an antibiotics solution; and
reacting the nanoparticle solution with the antibiotics solution, wherein the antibiotics is fixed on a surface of the nanoparticle by a covalent bond.

8. The method according to claim 7, wherein the nanoparticle is a metal nanoparticle, a nanoparticle with a core-shell structure, or an electroceramic nanocomposite.

9. The method according to claim 8, wherein the nanoparticle is a gold nanoparticle or a silica nanoparticle coated with a gold shell.

10. The method according to claim 7, wherein the antibiotics is vancomycin.

11. The method according to claim 10, wherein the vancomycin is the vancomycin dimer bearing a disulfide linkage.

12. A method of using the antibiotics according to claim 1 to treat bacteria infection, comprising administrating the antibiotics with an effective dose to a subject in need thereof, and exposing the subject under a near infrared radiation light source.

13. The method according to claim 12, wherein the near infrared radiation light source is a laser.

14. A pharmaceutical composition for treating bacteria infection, comprising the antibiotics according to claim 1 and a pharmaceutically acceptable carrier thereof.

15. The medicine combination according to claim 14, wherein the carrier is a solution, an excipient or an adhesive agent.

16. A method of using the antibiotics according to claim 14 to treat bacteria infection, comprising administrating the antibiotics with an effective dose to a subject in need thereof, and exposing the subject under a near infrared radiation light source.
